(19) United States
(12) Patent Application Publication
(10) Pub. No.: US 2025/0032371 A1
(43) Pub. Date: Jan. 30, 2025

(54) ETHANOL-FREE FRAGRANCE CHASSIS

(71) Applicant: Coty Inc., New York, NY (US)

(72) Inventor: Claudie Bellouard, Versoix (CH)

(21) Appl. No.: 18/704,769

(22) PCT Filed: Nov. 3, 2022
(86) PCT No.: PCT/US2022/079198

§ 371 Date: Apr. 25, 2024

Related U.S. Application Data
(60) Provisional application No. 63/275,811, filed on Nov. 4, 2021.

(51) Int. Cl. A61K 8/06; A61Q 13/00

(52) U.S. Cl. CPC A61K 8/068; A61Q 13/00; A61K 2800/21; A61K 2800/262; A61K 2800/30

(57) ABSTRACT

Various aspects according to the instant disclosure are related to an optically clear or transparent oil-in-water microemulsion that is substantially free (e.g., less than 1 wt %) of ethanol. Non-limiting examples of suitable fragrance components include a perfume, an Eaux de Toilette, an aqueous perfume, a body spray, or a body deodorant.

### CROSS-REFERENCE TO RELATED APPLICATION

This application claims the benefit of priority to U.S. Provisional Patent Application Ser. No. 63/275,811 entitled “ETHANOL-FREE FRAGRANCE CHASSIS,” filed Nov. 4, 2021, the disclosure of which is incorporated herein in its entirety by reference.

### BACKGROUND

The present disclosure relates to a transparent, ethanol-free perfumed aqueous microemulsion composition, such as fragrances, Eaux de Toilettes, body sprays, body deodorants, refreshing and cleaning wet towels, aqueous cosmetic compositions, household cleaners, and air fresheners.

### SUMMARY OF THE DISCLOSURE

Various aspects according to the instant disclosure are related to an optically clear or transparent oil-in-water microemulsion that is substantially free (e.g., less than 1 wt %) of ethanol. Non-limiting examples of suitable fragrance components include a perfume, an Eaux de Toilette, an aqueous perfume, a body spray, or a body deodorant.

According to various examples, the composition can be a component of a kit. The kit can include a container. A transparent oil-in-water microemulsion can be disposed within the container. The transparent oil-in-water microemulsion is an optically clear or transparent oil-in-water microemulsion that is substantially free (e.g., less than 1 wt %) of ethanol. Non-limiting examples of suitable fragrance components include a perfume, an Eaux de Toilette, an aqueous perfume, a body spray, or a body deodorant.

### DETAILED DESCRIPTION OF THE DISCLOSURE

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group. A hydrocarbylene group is a diradical hydrocarbon, e.g., a hydrocarbon that is bonded at two locations.

“Ethanol-free” when used herein in reference to a substance means substantially free of ethyl alcohol. “Substantially free” in this context means less than 3 wt %, preferably less than 1 wt %, and more preferably zero weight percent present in a substance.

A “microemulsion” as the term is used herein denotes a pseudo one-phase transparent mixture of (i) two immiscible fluids, and (ii) at least one amphiphile (exemplified by surfactants). Microemulsions are transparent or translucent, and do not display the opalescence of standard emulsions. The particle size of the resulting droplets is small enough so the resulting mixture is optically clear or translucent. Microemulsion droplet sizes are variously defined in the art with a droplet size typically below 0.14 micron. The clarity of these compositions is advantageous in cosmetic applications. Also, microemulsions are thermodynamically stable and form spontaneously.

For the purposes of the present disclosure, the term “optically clear” is used to define a composition that is “transparent” (e.g. transmitting light without distortion) which means that the size of the particles in the composition are reduced to a size where the particles are not observable with optical (visual) means. Transmitting light without distortion as used herein means being able to read 12-point text through a 1-centimeter thick sample of the microemulsion.

In the perfume industry there has been a long standing need for ethanol-free aqueous perfume compositions. It is well known to produce cosmetic compositions containing ethanol, by the consumers often designated as “alcohol”. Such ethanol-containing cosmetics are not acceptable for various reasons such as skin sensitivity, infant safety, and religious prohibition of use of ethanol for some consumers. Also, there is an ongoing trend to reduce the use of volatile organic chemicals (VOC). Aqueous compositions, on the other hand, have the benefits that water is environmentally friendly and the formulations are non-flammable. The perfume industry has attempted to develop ethanol-free compositions to meet the needs of the consumers.

Known ethanol-free perfume compositions, generally have the following drawbacks: the compositions are not always transparent; the compositions are sticky owing to the sticky nature of the surfactants and to the relatively large quantity of surfactants implemented; the perfume given off by the fragrance material present in the compositions is liable to be denatured by the solubilizing agent; the compositions are somewhat irritant owing to the irritant nature of the solubilizing agent and the aggressive nature of the surfactants; and the fragrance material present in the compositions are liable to be deteriorated by air oxidation, which detrimentally affects the stability and the olfactory characteristics of the compositions.

Various aspects according to the instant disclosure are related to an optically clear or transparent oil-in-water microemulsion that is substantially free (e.g., less than 1 wt %) of ethanol. Non-limiting examples of suitable fragrance components include a perfume, an Eaux de Toilette, an aqueous perfume, a body spray, or a body deodorant. According to various examples, a transparent oil-in-water microemulsion can include a fragrance component, a surfactant, a modulator that includes a glycol, water, and a transparency component. The ethanol-free, transparent, perfumed aqueous cosmetic microemulsion composition is generally non-greasy and non-sticky. It is particularly useful as a cosmetic, microemulsion composition.

According to various examples, the fragrance component is in a range of from about 0.5 wt % to about 15 wt % of the transparent oil-in-water microemulsion about 5 wt % to about 12 wt %, less than, equal to, or greater than about 0.5 wt %, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or about 15 wt % of the transparent oil-in-water microemulsion.

The transparent oil-in-water microemulsion can include any suitable component. For example, the transparent oil-in-water microemulsion can include musk oil, civet, castoreum, ambergris, plant fragrances such as nutmeg extract, cardomon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomile oil, clove oil, sage oil, neroli oil, labdanum oil, eucalyptus oil, verbena oil, mimosa extract, narcissus extract, carrot seed extract, jasmine extract, olibanum extract, rose extract, acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, benzyl acetate, benzyl alcohol and ester derivatives, benzyl propionate, benzyl salicylate, borneol, butyl acetate, camphor, carbitol, cinnamaldehyde, cinnamyl acetate, cinnamyl alcohol, cis-3-hexanol and ester derivatives, cis-3-hexenyl methyl carbonate, citral, citronellol and ester derivatives, cumin aldehyde, cyclamen aldehyde, cyclo galbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltolide, fenchone, fruity esters such as ethyl 2-methyl butyrate, galaxolide, geraniol and ester derivatives, helional, 2-heptonone, hexenol, α-hexylcinnamic aldehyde, hydroxycitronellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dihydrojasmonate, methyl eugenol, methyl ionone, methyl-β-naphthyl ketone, methylphenylcarbinyl acetate, mugetanol, γ-nonalactone, octanal, phenyl ethyl acetate, phenyl-acetaldehyde dimethyl acetate, phenoxyethyl isobutyrate, phenyl ethyl alcohol, pinenes, sandalore, santalol, stemone, thymol, terpenes, triplal, triethyl citrate, 3,3,5-trimethylcyclohexanol, γ-undecalactone, undecenal, vanillin, veloutone, verdox, or mixtures thereof.

The surfactant can be present in a range of from about 10 wt % to about 25 wt % of the transparent oil-in-water microemulsion, about 14 wt % to about 20 wt %, less than, equal to, or greater than about 10 wt %, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, or about 25 wt % of the transparent oil-in-water microemulsion. The surfactant can include many suitable components. For example the surfactant can include nonionic surfactants.

Suitable nonionic surfactants are disclosed in U.S. Pat. No. 3,929,678, Laughlin et al., issued Dec. 30, 1975, and U.S. Pat. No. 4,285,841, Barrat et al, issued Aug. 25, 1981. Exemplary, non-limiting classes of useful nonionic surfactants include C₈-C₁₈ alkyl ethoxylates, with about 1-22 ethylene oxide units, including the so-called narrow peaked alkyl ethoxylates and C₆-C₁₂ alkyl phenol alkoxylates, particularly ethoxylates and mixed ethoxylates/propoxylates, alkyl dialkyl amine oxides, alkanoyl glucose amides, and mixtures thereof. Other useful nonionic surfactants are polyethylene, polypropylene, and polybutylene oxide condensates of alkyl phenols. Commercially available nonionic surfactants of this type include Igepal® CO-630, marketed by the GAF Corporation; and Triton® X45, X-114, X-100, and X-102, all marketed by the Rohm & Haas Company. These compounds are commonly referred to as alkyl phenol alkoxylates, preferably alkyl phenol ethoxylates. Further useful nonionic surfactants are the condensation products of aliphatic alcohols with from about 1 to about 25 moles of ethylene oxide. The alkyl chain of the aliphatic alcohol can either be straight or branched, primary or secondary, and generally contains from about 8 to about 22 carbon atoms. Examples of commercially available nonionic surfactants of this type include Tergitol® 15-S-9 (the condensation product of C₁₁-C₁₅ linear secondary alcohol with 9 moles ethylene oxide), Tergitol® 24-L-6 NMW (the condensation product of C₁₂-C₁₄ primary alcohol with 6 moles ethylene oxide with a narrow molecular weight distribution), both marketed by The Dow Chemical Corporation; Neodol® 45-9 (the condensation product of C₁₄-C₁₅ linear alcohol with 9 moles of ethylene oxide), Neodol® 23-6.5 (the condensation product of C₁₂-C₁₃ linear alcohol with 6.5 moles of ethylene oxide), Neodol® 45-7 (the condensation product of C₁₄-C₁₅ linear alcohol with 7 moles of ethylene oxide), Neodol® 45-4 (the condensation product of C₁₄-C₁₅ linear alcohol with 4 moles of ethylene oxide), marketed by Shell Chemical Company, and Kyro® EOB (the condensation product of C₁₃-C₁₅ alcohol with 9 moles ethylene oxide), marketed by The Procter & Gamble Company. Other commercially available nonionic surfactants include Dobanol 91-8® marketed by Shell Chemical Co. and Genapol UD-080® marketed by Hoechst. This category of nonionic surfactant is referred to generally as “alkyl ethoxylates.” Other useful nonionic surfactants are the condensation products of ethylene oxide with a hydrophobic base formed by the condensation of propylene oxide with propylene glycol. Examples of compounds of this type include certain of the commercially-available Pluronic® surfactants, marketed by BASF. Further useful surfactants are the condensation products of ethylene oxide with the product resulting from the reaction of propylene oxide and ethylenediamine. Examples of this type of nonionic surfactants include certain of the commercially available Tetronic® compounds, marketed by BASF. Semi-polar nonionic surfactants are a special category of nonionic surfactants which include water-soluble amine oxides. These amine oxide surfactants in particular include C₁₀-C₁₈ alkyl dimethyl amine oxides and C₈-C₁₂ alkoxy ethyl dihydroxy ethyl amine oxides. Other nonionic surfactants are alkylpolysaccharides. Any reducing saccharide containing 5 or 6 carbon atoms can be used, e.g., glucose, galactose and galactosyl moieties can be substituted for the glucosyl moieties. Fatty acid amide surfactants, C₁₂-C₁₈ betaines and sulfobetaines (sultaines) are also knows surfactants.

Further useful surfactants are amine oxide surfactants. Commercially available amine oxides are the solid, dihydrate ADMOX 16 and ADMOX 18, ADMOX 12 and especially ADMOX 14 from Ethyl Corp. Other surfactants include dodecyldimethylamine oxide dihydrate, hexadecyldimethylamine oxide dihydrate, octadecyldimethylamine oxide dihydrate, hexadecyltris(ethyleneoxy)dimethyl-amine oxide, tetradecyldimethylamine oxide dihydrate, and mixtures thereof.

Other useful surfactants are biodegradably branched surfactants are more fully disclosed in WO98/23712 A published Jun. 4, 1998; WO97/38957 A published Oct. 23, 1997; WO97/38956 A published Oct. 23, 1997; WO97/39091 A published Oct. 23, 1997; WO97/39089 A published Oct. 23, 1997; WO97/39088 A published Oct. 23, 1997; WO97/39087 A1 published Oct. 23, 1997; WO97/38972 A published Oct. 23, 1997; WO 98/23566 A Shell, published Jun. 4, 1998.

Specifically useful surfactants can include a polyglyceryl fatty acid ester, polyglyceryl-3 diisostearate, polyglyceryl-4 isostearate, polyglyceryl-3 cocoate, polyglyceryl-6 Ricinoleate, polyglyceryl-3 stearate polyglyceryl-10 stearate, polyglyceryl-10 oleate, polyglyceryl-4 diisostearate/polyhydroxystearate/sebacate, polyglyceryl-2 dipolyhydroxystearate, polyglyceryl-3 poliricinoleate, polyglyceryl-6 stearate (and) polyglyceryl 6-behenate, sorbitan esters, sucrose esters, alkyl polyglycosides, fatty acid glucamides, or a mixture thereof. If the surfactant includes a polyglyceryl fatty acid ester, the polyglyceryl fatty acid ester can include a mixture of polyglyceryl-4 laurate/sebacate; polyglyceryl-6 caprylate/caprate; and water. The polyglyceryl fatty acid ester can alternatively or additionally include a mixture of polyglyceryl-4 laurate/sebacate, polyglyceryl-4 caprylate/caprate; and water. The polyglyceryl fatty acid ester can alternatively or additionally include polyglyceryl-4 caprate.

The modulator can be present in a range of from about 1 wt % to about 15 wt % of the transparent oil-in-water microemulsion, about 3 wt % to about 10 wt %, less than, equal to, or greater than about 1 wt %, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or about 15 wt % of the transparent oil-in-water microemulsion. The modulator can include glycol or polycitronellol. As a specific example, the glycol can be pentylene glycol, glucam, polycitronellol, or a mixture thereof.

In other examples, instead of or in addition to glycol or polycitronellol, suitable examples of other modulators include:
the compound according to Formula I:

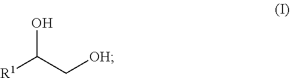

a polymer including a repeating unit derived from the compound according to Formula II:

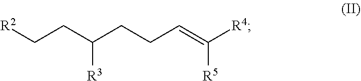

a mixture thereof. R¹, R², R³, R⁴, and R⁵, are independently chosen from —H, —OH, or substituted or unsubstituted (C₁-C₂₀)hydrocarbyl. In some further embodiments, R¹, R², R³, R⁴, and R⁵, are independently chosen from (C₁-C₂₀)alkyl, (C₂-C₂₀)alkeneyl, (C₃-C₂₀)cycloalkyl, (C₁-C₂₀)alkoxyl, (C₁-C₂₀)aryl, or a combination thereof. In some specific embodiments, the modulator component includes pentylene glycol, polycitronellol, or a mixture thereof. In some embodiments, the polycitronellol can include 2-8 repeating units and can have a weight-average-molecular weight in a range of from about 460 g/mol to about 1500 g/mol. In some embodiments, the modulator component can include a mixture of pentylene glycol and polycitronellol and a molar ratio of pentylene glycol to polycitronellol is in a range of from about 5:1 to about 1:5, about 4:1 to about 1:4, about 3:1 to about 1:3, or about 2:1 to about 1:2, or about 1:1. In some embodiments, the modulator component can include at least some PPG-20 methyl glucose ether mixed with any of the aforementioned modulators. Alternativly, the modulator component can be free of (include 0 wt %) PPG-20 methyl glucose. If PPG-20 methyl glucose is present, it can be in range of from about 1 wt % to about 15 wt % of the modulator component, about 5 wt % to about 10 wt % of the modulator component, less than, equal to, or greater than about 1 wt %, 2, 3, 4, 5, 6, 7, 8, 9, 10, 11, 12, 13, 14, or about 15 wt %.

In some further examples, the modulator component can include other modulators included in addition to pentylene glycol, polycitronellol, or a mixture thereof. Examples of the additional modulators can include those listed herein below in Tables 4(a) and 4(b).

Tables 4(a) and 4(b) provide lists of suitable non-odorous modulators.

TABLE 4(a)
Substantially Non-Odorous Modulators

| | | | CAS | |
|---|---|---|---|---|
| No. | Group | Chemical Name | Number | Supplier |
| 1. | (a) | PPG-10 Methyl Glucose Ether | 61849-72-7 | Lubrizol |
| 2. | | PPG-20 Methyl Glucose Ether ¹ | 61849-72-7 | |
| 3. | | Ethoxylated Methyl Glucose | 68239-42-9 | |
| | | Ether ² | | |
| 4. | | Caprylyl/Capryl Glucoside ³ | 68515-73-1 | BASF |
| 5. | | Undecyl Glucoside ^{3a} | — | SEPPIC |
| | | | | (France) |
| 6. | (b) | Isocetyl Alcohol ⁴ | 36653-82-4 | Ashland |
| | | | | Speciality |
| | | | | Ingredients |
| 7. | (c) | PPG-3 Myristyl Ether ⁵ | — | Evonik |
| 8. | | Neopentyl Glycol | 28510-23-8 | Lubrizol |
| | | Diethylhexanoate ⁶ | | |
| 9. | (d) | Sucrose Laurate | 25339-99-5 | Alfa |
| | | | | Chemicals |
| | | | | Ltd. (UK) |
| 10. | | Sucrose dilaurate | 25915-57-5 | Alfa |
| | | | | Chemicals |
| | | | | Ltd. (UK) |
| 11. | | Sucrose Myristate | 27216-47-3 | Mitsubishi |
| | | | | Chemicals |
| 12. | | Sucrose Palmitate | 26446-38-8 | Alfa |
| | | | | Chemicals |
| 13. | | Sucrose Stearate | 25168-73-4 | Ltd. (UK) |
| 14. | | Sucrose Distearate | 27195-16-0 | Mitsubishi |
| | | | | Chemicals |
| | | | | (JP) |
| 15. | | Sucrose Tristearate | 27923063-3 | Mitsubishi |
| | | | | Chemicals |
| | | | | (JP) |
| 16. | (e) | (E)-1-(2,2,6- | — | Takasago |
| | | trimethylcyclohexyl)oct-1-en-3- | | (Japan) |
| | | one ⁸ | | |
| 17. | (f) | 2-(1-menthoxy)ethane-1-ol ⁹ | — | Takasago |
| 18. | | 1-(1-menthoxy)propane-2-ol ⁹ | — | (Japan) |
| 19. | | 3-(1-menthoxy)propane-1-ol ⁹ | — | |
| 20. | | 3-(1-menthoxy)propane-1,2- | — | |
| | | diol ⁹ | | |
| 21. | | 2-methyl-3-(1- | — | |
| | | menthoxy)propane-1,2-diol ⁹ | | |
| 22. | | 4-(1-menthoxy) butane-1-ol ⁹ | — | |
| 23. | (g) | 1,1,4,4-tetramethyl-6-acetyl-7- | — | Givaudan |
| | | formyl-1,2,3,4- | | (Switzerland) |
| | | tetrahydronaphthalene ¹⁰ | | |
| 24. | | 1,1,2,4,4-pentamethyl-6-acetyl- | — | |
| | | 7-formyl-1,2,3,4- | | |
| | | tetrahydronaphthalene ¹⁰ | | |
| 25. | (h) | Hyaluronic acid disaccharide | 9004-61-9 | |
| | | sodium salt 11 | | |
| 26. | | Sodium Hyaluronate ¹¹ | 9067-32-7 | Sigma |
| | | | | Aldrich |
| | | | | (UK) |
| 27. | (i) | Mono-o-(linalyl)-glucopyranose ¹² | — | Kanebo |
| 28. | | Di-o-(linalyl)-glucopyranose ¹² | — | (Japan) |
| 29. | | Tri-o-(linalyl)-glucopyranose ¹² | — | |
| 30. | | Tetra-o-(linalyl)-glucopyranose ¹² | — | |
| 31. | | Penta-o-(linalyl)-glucopyranose ¹² | — | |
| 32. | | Mono-o-(cis-3-hexenyl)- | — | |
| | | glactopyranose ¹² | | |
| 33. | | Di-o-(cis-3-hexenyl)- | — | |
| | | glactopyranose ¹² | | |
| 34. | | Tri-o-(cis-3-hexenyl)- | — | |
| | | glactopyranose ¹² | | |
| 35. | | Tetra-o-(cis-3-hexenyl)- | — | |
| | | glactopyranose ¹² | | |
| 36. | | Penta-o-(cis-3-hexenyl)- | — | |
| | | glactopyranose ¹² | | |
| 37. | (j) | Bis-O-(3,6-dioxadecanyl)- | — | |
| | | glucopyranose ¹³ | | |
| 38. | | Tris-O-(3,6-dioxadecanyl)- | — | |
| | | glucopyranose ¹³ | | |
| 39. | | Tetrakis-O-(3,6-dioxadecanyl)- | — | |
| | | glucopyranose ¹³ | | |
| 40. | | Pentakis-O-(3,6-dioxadecanyl)- | — | |
| | | glucopyranose¹³ | | |
| 41. | | Bis-O-(3,6-dioxaoctanyl)- | — | |
| | | galactopyranose ¹³ | | |
| 42. | | Tris-O-(3,6-dioxaoctanyl)- | — | |
| | | galactopyranose ¹³ | | |
| 43. | | Tetrakis-O-(3,6-dioxaoctanyl)- | — | |
| | | galactopyranose¹³ | | |
| 44. | | Pentakis-O-(3,6-dioxaoctanyl)- | — | |
| | | galactopyranose ¹³ | | |
| 45. | | Bis-O-(3,6-dioxaheptanyl)- | — | |
| | | xylopyranose ¹³ | | |
| 46. | | Tris-O-(3,6-dioxaheptanyl)- | — | |
| | | xylopyranose ¹³ | | |
| 47. | | Tetrakis-O-(3,6-dioxaheptanyl)- | — | |
| | | xylopyranose ¹³ | | |
| 48. | | Bis-O-(3,6-dioxadodecanyl)- | — | |
| | | glucopyranose ¹³ | | |
| 49. | | Tris-O-(3,6-dioxadodecanyl)- | — | |
| | | glucopyranose ¹³ | | |
| 50. | | Tetrakis-O-(3,6- | — | |
| | | dioxadodecanyl)-glucopyranose ¹³ | | |
| 51. | | Pentakis-O-(3,6- | — | |
| | | dioxadodecanyl)- | | |
| | | glucopyranose ¹³ | | |
| 52. | (k) | Hydroquinone beta-D-glycoside ¹⁴ | 497-76-7 | Shiseido |
| 53. | (1) | Propylene Glycol Propyl Ether | 1569-01-3 | Sigma |
| | | | | Aldrich |
| 54. | | Dicetyl Ether | 4113-12-6 | (UK) |
| 55. | | Polyglycerin-4 Ethers | 25618-55-7 | Solvay |
| | | | | Chemicals |
| 56. | | Isoceteth-5 | 69364-63-2 | Nihon |
| 57. | | Isoceteth-7 | 69364-63-2 | Emulsion |
| 58. | | Isoceteth-10 | 69364-63-2 | Company |
| 59. | | Isoceteth-12 | 69364-63-2 | Ltd. |
| 60. | | Isoceteth-15 | 69364-63-2 | |
| 61. | | Isoceteth-20 | 69364-63-2 | |
| 62. | | Isoceteth-25 | 69364-63-2 | |
| 63. | | Isoceteth-30 | 69364-63-2 | |
| 64. | | Disodium | 68929-04-4 | Rhodia |
| | | Lauroamphodipropionate | | |
| 65. | | Hexaethylene glycol | 3055-96-7 | Sigma |
| | | monododecyl ether ^{14b} | | Aldrich |
| | | | | (UK) |
| 66. | (m) | Neopentyl Glycol | 27841-07-2 | Symrise |
| | | Diisononanoate ¹⁵ | | (Germany) |
| 67. | | Cetearyl Ethylhexnoate ¹⁶ | 90411-68-0 | |
| 68. | (n) | 2-ethylhexyloxypropanediol ¹⁷ | 70455-33-9 | Takasago |
| | | | | (JP) |
| 69. | (o) | Panthenol Ethyl Ether ¹⁸ | 667-83-4 | DSM |
| | | | | Nutritional |
| | | | | Products, |
| | | | | Inc. (USA) |
| 70. | | DL-Panthenol | 16485-10-2 | Roche Inc. |
| | | | | (USA) |
| 71. | (p) | Diisobutyl Adipate ¹⁹ | 141-04-8 | Sigma |
| | | | | Aldrich |
| 72. | | Diisoamyl Adipate ¹⁹ | 6624-70-0 | (UK) |
| 73. | (q) | PPG-11 Stearyl Ether ^{19a} | 25231-21-4 | Kao (JP) |
| 74. | (r) | N-hexadecyl n-nonanoate ^{19b} | 72934-15-7 | Symrise |
| | | (e.g., cetyl nonanoate) | | (Germany) |
| 75. | | Noctadecyl n-nonanoate ^{19b} | 107647-13-2 | |
| | | (e.g., stearyl nonanoate) | | |
| 76. | (s) | methanone, (morphonyl) | — | Unilever |
| | | tricyclo[3.3.1.1^{3,7}]dec-1-yl- ²⁰ | | (UK) |
| 77. | | methanone, (piperidinyl) | — | |
| | | tricyclo[3.3.1.1^{3,7}]dec-1-yl- ²⁰ | | |
| 78. | | methanone, (pyrrolidinyl) | — | |
| | | tricyclo[3.3.1.1^{3,7}]dec-1-yl ²⁰ | | |
| 79. | | methanone, (azetidinyl) | — | |
| | | tricyclo[3.3.1.1^{3,7}]dec-1-yl- ²⁰ | | |
| 80. | | methanone, | — | |
| | | (hexahydroazepinyl) | | |
| | | tricyclo[3.3.1.1^{3,7}]dec-1-yl- ²⁰ | | |
| 81. | | methanone, (4-cyano- | — | |
| | | piperidinyl)tricyclo[3.3.1.1^{3,7}]dec-1-yl- ²⁰ | | |
| 82. | | methanone, (4-amido- | — | |
| | | piperidinyl)tricyclo[3.3.1.1^{3,7}]dec-1-yl- ²⁰ | | |
| 83. | | methanone, | — | |
| | | (Tricyclo[3.3.1.1^{3,7}]decanyl)-N- | | |
| | | tricyclo[3.3.1.1^{3,7}]dec-1-yl- ²⁰ | | |
| 84 | | methanone, | — | |
| | | (decahydroisoquinolinyl)tricyclo[3.3.1.1^{3,7}]dec-1-yl- ²⁰ | | |
| 85. | | methanone, | — | |
| | | (decahydroisoquinolinyl)tricyclo[3.3.1.1^{3,7}]dec-1-yl- ²⁰ | | |
| 86. | | methanone, | — | |
| | | (decahydroquinolinyl)tricyclo[3.3.1.1^{3,7}]dec-1-yl- ²⁰ | | |
| 87. | | methanone, (3,3-dimethyl-1- | — | |
| | | piperidinyl)tricyclo[3.3.1.1^{3,7}] | | |
| | | dec-1-yl- ²⁰ | | |
| 88. | | methanone, (2-methyl-1- | — | |
| | | piperidinyl)tricyclo[3.3.1.1^{3,7}] | | |
| | | dec-1-yl- ²⁰ | | |
| 89. | | methanone, (4-methyl-1- | — | |
| | | piperidinyl)tricyclo[3.3.1.1^{3,7}] | | |
| | | dec-1-yl- ²⁰ | | |
| 90. | | methanone, (3-methyl-1- | — | |
| | | piperidinyl)tricyclo[3.3.1.1^{3,7}] | | |
| | | dec-1-yl- ²⁰ | | |
| 91. | | methanone, (3,5-dimethyl-1- | — | |
| | | piperidinyl)tricyclo[3.3.1.1^{3,7}] | | |
| | | dec-1-yl- ²⁰ | | |
| 92. | | methanone, (4-methyl-4-ethy- | — | |
| | | piperidinyl)tricyclo[3.3.1.1^{3,7}] | | |
| | | dec-1-yl- ²⁰ | | |
| 93. | | methanone, (3,3-diethyl-1- | — | |
| | | pyrrolidinyl)tricyclo[3.3.1.1^{3,7}] | | |
| | | dec-1-yl- ²⁰ | | |
| 94. | | methanone, (N,N-diisopropyl) | — | |
| | | tricyclo[3.3.1.1^{3,7}]dec-1-yl- ²⁰ | | |
| 95. | | methanone, (3,3- | — | |
| | | dimethylbutylaminyl) | | |
| | | tricyclo[3.3.1.1^{3,7}]dec-1-yl- ²⁰ | | |
| 96. | | methanone, (2,2- | — | |
| | | dimethylpropylaminyl) | | |
| | | tricyclo[3.3.1.1^{3,7}]dec-1-yl- ²⁰ | | |
| 97. | | methanone, (1,1-dimethyl-3,3- | — | |
| | | dimethylbutylaminyl) | | |
| | | tricyclo[3.3.1.1^{3,7}]dec-1-yl- ²⁰ | | |
| 98. | | methanone, (1,3-dimethyl- | — | |
| | | butylaminyl) | | |
| | | tricycle[3.3.1.1^{3,7}]dec-1-yl- ²⁰ | | |
| 99. | (t) | Bis-methoxy PEG-13 PEG- | 936645-35-1 | PolymerExpert |
| | | 438/PPG-110 SMDI Copolymer ²¹ | | S.A. |
| | | | | (Pessac, |
| | | | | France) |
| 100. | (u) | propyl {4-[2-(diethylamino)-2- | 61791-12-6 | Sigma |
| | | oxoethoxy]-3- | | Aldrich |
| | | methoxyphenyl}acetate ²² | | (US) |
| 101. | | 3-((2-ethylhexyl)oxy)propane- | 70445-33-9 | — |
| | | 1,2-diol ²³ | | |
| 102. | (v) | 3-((2- | | — |
| | | propylheptyl)oxy)propane-1,2- | | |
| | | diol ²³ | | |
| 103. | | 1-amino-3-((2- | 99509-00-9 | — |
| | | ethylhexyl)oxy)propan-2-ol ²³ | | |

¹ available as GLUCAM ™ P-20.
² available as Glucam ™ E-20.
³ available as Plantacare ® 810 UP.
^{3a} available as Simulsol ® SL 11W.
⁴ available as CERAPHYL ® ICA.
⁵ available as Tegosoft ® APM.
⁶ available as Schercemol ™ NGDO.
⁷ disclosed in U.S. Pat. No. 6,737,396B2 (Firmenich), column 1, lines 43-47.
⁸ diclosed as compound 1′i in U.S. Pat. No. 6,440,400B1 (Takasago Int. Corp.), col. 5.
^{8a} diclosed in U.S. Pat. No. 4,313,855 (Dragoco Gerberding & Co. GmbH), col. 1, lines 12-13.
⁹ disclosed in U.S. Pat. No. 7,538,081B2 (Takasago Int. Corp.), column 7, lines 50-53.
¹⁰ disclosed in U.S. Pat. No. 6,147,049 (Givaudan Roure), col. 5, line 24, to col. 6, line 17.
¹¹ disclosed in PCT Publication No. WO85/04803 (Diagnostic), pg. 2, line 1 to pg. 4, line 2.
¹² disclosed in JP Patent No. 61-083114 (Kanebo).
¹³ disclosed in JP Patent No. 61-063612 (Kanebo).
¹⁴ disclosed in JP Patent No. 62-084010 (Shiseido).
^{14b} available as: Laureth-6.
¹⁵ disclosed in U.S. Patent Publication No. 2011/0104089A1 (Symrise), para. [0001].
¹⁶ available as PCL-Liquid ® 100.
¹⁷ disclosed in U.S. Pat. No. 7,196,052 (Takasago Int. Corp.), col. 4, lines 34-35.
¹⁸ disclosed in EP Patent Publication No. 616800A2 (Givaudan), pg. 2, lines 12-25.
¹⁹ disclosed in U.S. Pat. No. 4,110,626 (Shiseido), column 3, lines 54-56.
^{19a} disclosed in PCT Publication No. WO2014/155019 (LVMH).
^{19b} disclosed in U.S. Pat. No. 9,050,261 (Symrise).
²⁰ disclosed as compounds C1-C22 in WO2014/139952 (Unilever).
²¹ available as Expert Gel ® EG56.
²² available as Kolliphor ® EL.
²³ disclosed in U.S. Pat. No. 9,050,261 (Symrise).

Further examples of non-odorous modulators that can be included instead of or in addition to the modulators described herein can include one that is selected from the group of materials disclosed in Table 4(b).

TABLE 4b
Substantially Non-Odorous Modulators

| No. | Chemical or INCI Name | Trade Name | CAS Number | Supplier |
|---|---|---|---|---|
| 1. | C12-14 Sec-Pareth-3 | Tergitol ® 15-S-7 | 68131-40-8 | Sigma Aldrich (UK) |
| 2. | Poly(ethylene glycol- | PPG-7-Buteth-10 | 9038-95-3 | Sigma Aldrich (UK) |
| | ran-propylene glycol) | | | |
| | monobutyl ether | | | |
| 3. | PPG-4-Ceteth-10 | Nikkol PBC-33 | 37311-01-6 | Chemical Navi |
| 4. | Deceth-4 | Ethal DA-4 | 5703-94-6 | Ethox Chemicals, Inc. |
| 5. | PPG-5-Ceteth-20 | AEC PPG-5-Ceteth-20 | 9087-53-0 | A & E Connock (Perfumery |
| | | | | & Cosmetics) Ltd. |
| 6. | C14-15 Pareth-7 | Neodol 45-7 alcohol | 68951-67-7 | Shell Chemical Company |
| | | ethoxylate | | |
| 7. | Linear alcohol (C12- | Bio-soft N25-7 | 68131-39-5 | Stephan Company (USA) |
| | 15) Pareth-3ethoxylate, | | | |
| | POE-7 | | | |
| 8. | Linear alcohol (C12- | Bio-soft N23-6.5 | 66455-14-9 | |
| | 13) Pareth-3ethoxylated, | | | |
| | POE-6.5) | | | |
| 9. | Polyethylene glycol 1100 | Cremophor ® A 25 | 68439-49-6 | Sigma Aldrich (UK) |
| | mono(hexadecyl/octadecyl) | | | |
| | ether | | | |
| 10. | Linear alcohol (C9-11) | Bio-soft N91-8 | 68439-46-3 | Stephan Company (USA) |
| | ethoxylated POE-8 | | | |
| | Pareth-3 | | | |
| 11. | Coceth-10 or | Genapol ® C-100 | 61791-13-7 | Sigma Aldrich (UK) |
| | Polyoxyethylene (10) | | | |
| | dodecyl ether | | | |
| 12. | Alcohols, C12-14, | Rhodasurf ® LA 30 | 68439-50-9 | Solvay Solutions |
| | ethoxylated | | | Italia S.p.A. |
| 13. | Poly(ethylene glycol) | Poly(ethylene glycol) | 9004-74-4 | Sigma Aldrich (UK) |
| | methyl ether | methyl ether | | |
| 14. | C10-16 Pareth-1 | Neodol ® PC 110 | 68002-97-1 | Shell Chemical Company |
| 15. | PPG-11 Stearyl Ether | Arlamol ™ PS11E | 25231-21-4 | Croda (UK) |
| 16. | Steareth-100 | Brij ® S100 | 9005-00-9 | Sigma Aldrich (UK) |
| 17. | Polyethylene glycol | Brij ® C-58 | 9004-95-9 | Sigma Aldrich (UK) |
| | hexadecyl ether | | | |
| 18. | Pluronic ® F-127 | Pluronic ® F-127 | 9003-11-6 | Sigma Aldrich (UK) |
| 19. | Linear Alcohol (C11) | Bio-soft N1-5 | 34398-01-1 | Stepan Canada Inc. |
| | Ethoxylate, POE-5 | | | |
| 20. | Laureth-10 | Intrasol FA | 6540-99-4 | Evonik Industries AG |
| | | 12/18/10 | | |
| 21. | Decaethylene glycol | Polyoxyethylene | 9002-92-0 | Sigma Aldrich (UK) |
| | mono-dodecyl ether | (10) lauryl ether | | |
| 22. | Ethylene glycol monomethyl ether | 2-Methoxyethanol | 109-86-4 | Sigma Aldrich (UK) |
| 23. | Myreth-4 | Homulgator 920 G | 27306-79-2 | Grau Aromatics GmbH & Company KG |
| 24. | Oleth-16 Alkoxylated Alcohols | Pegnol O-16A | 25190-05-0 | Toho Chemical Industry Co., Ltd. |
| 25. | Isosteareth-5 | Emalex 1805 | 52292-17-8 | Nihon Emulsion Company, Ltd. |
| 26. | PPG-10 Cetyl Ether | Arlamol ™ PC10 | 9035-85-2 | Croda (UK) |
| 27. | Polyoxy(ethylene glycol) | Poly(ethylene glycol) | 24938-91-8 | Sigma-Aldrich (UK) |
| | (18) tridecyl ether | (18) tridecyl ether | | |
| 28. | Poly(oxy-1,2-ethanediyl), | ALFONIC ® 10-8 | 26183-52-8 | Sasol Chemicals (USA) LLC |
| | a-decyl-w-hydroxy- | Ethoxylate | | |
| 29. | Laureth- 1 | Mackam ™ 2LSF | 4536-30-5 | Rhodia (DE) |
| 30. | PEG-5 Hydrogenated Tallow Amine | Ethox HTAM-5 | 61791-26-2 | Ethox Chemicals, Inc. |
| 31. | PEG-15 Oleamine | Nikkol TAMNO-15 | 26635-93-8 | Nikko Chemicals Co., Ltd. |
| 32. | Polyoxyethylene (20) oleyl ether | Brij ® O20-SS | 9004-98-2 | Sigma Aldrich (UK) |
| 33. | Cetoleth-10 | Brij ® CO10 | 8065-81-4 | Croda, Inc. |
| 34. | Talloweth-7 | Emulmin 70 | 61791-28-4 | Sanyo Chemical Industries Ltd. |
| 35. | Isobutoxypropanol Alcohols | Isobutoxypropanol | 34150-35-1 | MolPort |
| 36. | Isobutoxypropanol Alcohols | Isobutoxypropanol | 23436-19-3 | AKos Consulting & Solutions |
| 37. | Diethylene Glycol | Twincide EDG | 111-46-6 | Roda |
| 38. | Methoxyethanol | Hisolve MC | 109-86-4 | Toho Chemical Industry Co., Ltd. |
| 39. | Ethoxyethanol Alcohols | 2-Ethoxyethanol | 110-80-5 | Sigma-Aldrich (UK) |
| 40. | Methoxyisopropanol Alcohols | Dowanol ™ PM | 107-98-2 | The Dow Chemical Company |
| 41. | Methoxyethanol | Hisolve MC | 32718-54-0 | Toho Chemical Industry Co., Ltd. |
| 42. | Methylal Ethers | Dimethoxymethane | 109-87-5 | Sigma-Aldrich (UK) |
| 43. | 3-Methoxybutanol | Methoxybutanol | 2517-43-3 | Hans Schwarzkopf GmbH/Co. KG |
| 44. | Butoxyethanol | Butyl OXITOL | 111-76-2 | Shell Chemical Company |
| 45. | Propylene Glycol n-Butyl Ether | Dowanol ™ PnB | 5131-66-8/ | The Dow Chemical Company |
| | | | 29387-86-8 | |
| 46. | Propylene Glycol Butyl Ether | Propylene Glycol | 15821-83-7 | Sigma Aldrich (UK) |
| | | Butyl Ether | | |
| 47. | 2-(2-butoxyethoxy)ethanol | Diethylene glycol | 112-34-5 | Sigma Aldrich (UK) |
| | | butyl ether | | |
| 48. | Deceth-4 Phosphate | Crodafos ™ D4A | 52019-36-0 | Croda, Inc. |
| 49. | 2-(Hexadecyloxy)ethanol | Ethylene glycol | 2136-71-2 | Sigma-Aldrich (UK) |
| | | monohexadecyl ether | | |
| 50. | Poly(propylene glycol) | Poly(propylene glycol) | 9003-13-8 | Sigma-Aldrich (UK) |
| | monobutyl ether | monobutyl ether | | |
| 51. | Propylene Glycol | Dowanol ™ PnP | 30136-13-1 | The Dow Chemical Company |
| | Propyl Ether | | | |
| 52. | Propylene Glycol n- | Dowanol ™ PnB | 29387-86-8/ | The Dow Chemical Company |
| | Butyl Ether | | 5131-66-8 | |
| 53. | Dipropylene glycol | Di(propylene glycol) | 34590-94-8 | Sigma Aldrich (UK) |
| | monomethyl ether | methyl ether, mixture | | |
| | | of isomers | | |
| 54. | Dipropylene Glycol | Proglyde ™ DMM | 111109-77-4 | The Dow Chemical Company |
| | Dimethyl Ether | | | |
| 55. | PPG-2 Methyl Ether | Dowanol ™ DPM | 13429-07-7 | The Dow Chemical Company |
| 56. | Methoxydiglycol Ethers | OriStar DEGME | 111-77-3 | Orient Stars LLC |
| 57. | Diethylene glycol ethyl | Di(ethylene glycol) | 111-90-0 | Sigma Aldrich (UK) |
| | ether | ethyl ether | | |
| 58. | Dimethoxydiglycol | Dimethyldiglycol | 111-96-6 | H&V Chemicals |
| | Ethers | | | |
| 59. | PPG-3 Methyl Ether | Dowanol ™ TPM | 37286-64-9 | The Dow Chemical Company |
| 60. | Methyl Morpholine | 224286 ALDRICH | 7529-22-8 | Sigma-Aldrich (UK) |
| | Oxide Amine Oxides | 4-Methylmorpholine | | |
| | | N-oxide | | |
| 61. | Oleth-3 | Brij ® 03 | 5274-66-8 | Croda Europe, Ltd. |
| 62. | Tri(propylene glycol) | Dowanol ™ TPnB | 55934-93-5 | Sigma-Aldrich (UK) |
| | n-butyl ether | | | |
| 63. | Tripropylene Glycol | Tripropylene Glycol | 24800-44-0 | Sigma-Aldrich (UK) |
| 64. | PPG-3 Methyl Ether | Dowanol ™ TPM | 25498-49-1 | The Dow Chemical Company |
| | Alkoxylated Alcohols | | | |
| 65. | Triethylene glycol | Triglycol | 112-27-6 | Sigma Aldrich (UK) |
| 66. | PEG-3 Methyl Ether | Hymol ™ | 112-35-6 | Toho Chemical Industry Co., Ltd. |
| 67. | Laureth-3 | AEC Laureth-3 | 3055-94-5 | A & E Connock (Perfumery & |
| | | | | Cosmetics) Ltd. |
| 68. | Ethylhexylglycerin | AG-G-75008 | 70445-33-9 | Angene Chemical |
| 69. | Tetra(ethylene glycol) | Tetraethylene glycol | 112-60-7 | Sigma Aldrich (UK) |
| 70. | Steareth-3 | Isoxal 5 | 4439-32-1 | Vevy Europe SpA |
| 71. | Ceteth-3 | Emalex 103 | 4484-59-7 | Nihon Emulsion Company, Ltd. |
| 72. | Myreth-3 | Isoxal 5 | 26826-30-2 | Vevy Europe SpA |
| 73. | Trideceth-3 | Alfonic ® TDA- | — | Sasol North America, Inc. |
| | | 3 Ethoxylate | | |
| 74. | Ceteth-2 | Brij ® C2 | 5274-61-3 | Croda Europe, Ltd. |
| 75. | Oleth-2 | Brij ® O2 | 5274-65-7 | Croda, Inc. |
| 76. | Steareth-2 | Brij ® S2 | 16057-43-5 | Croda, Inc. |
| 77. | Cetoleth-10 | Brij ® CO10 | 8065-81-4 | Croda, Inc. |
| 78. | Trimethyl Pentanol Hydroxyethyl | Trimethyl Pentanol | 68959-25-1 | Angene Chemical |
| | Ether Alcohols | Hydroxyethyl Ether | | |
| 79. | Steareth-10 Allyl Ether | Salcare ® SC80 | 109292-17-3 | BASF |
| 80. | TEA-Lauryl Ether | material ID-AG-J-99109 | 1733-93-3 | Angene Chemical |
| 81. | Polyglyceryl-2 Oleyl Ether | Chimexane NB | 71032-90-1 | Chimex |
| 82. | Batyl Alcohol | B402 ALDRICH | 544-62-7 | Sigma-Aldrich (UK) |
| 83. | Octaethylene Glycol | 15879 ALDRICH | 5117-19-1 | Sigma-Aldrich (UK) |
| 84. | Triglycerol diisostearate | Cithrol ™ | 66082-42-6 | Croda (UK) |
| 85. | Diglycerin | Diglycerin 801 | 59113-36-9 | Sakamoto Yakuhin Kogyo Co., Ltd. |
| 86. | Polyglycerin #310 | Polyglycerin #310 | 25618-55-7 | Sakamoto Yakuhin Kogyo Co., Ltd. |
| 87. | Distearyl Ether | Cosmacol ® SE | 6297-03-6 | Sasol Germany GmbH |
| 88. | Caprylyl Glyceryl Ether | Caprylyl Glyceryl Ether | 10438-94-5 | AKos Consulting & Solutions |
| 89. | Chimyl Alcohol | Chimyl Alcohol | 506-03-6 | Nikko Chemicals Co., Ltd. |
| 90. | Dipentaerythrityl | Liponate ® DPC-6 | 68130-24-5 | Lipo Chemicals, Inc. |
| | Hexacaprylate/Hexacap rate | | | |
| 91. | Morpholine | 394467 ALDRICH | 110-91-8 | Sigma-Aldrich (UK) |
| 92. | Dimethyl Oxazolidine | OXABAN ™ -A | 51200-87-4 | The Dow Chemical Company |
| 93. | Ethyl Hydroxymethyl | 4-Oxazolemethanol | 68140-98-7 | Angene Chemical |
| | Oleyl Oxazoline | | | |
| 94. | Methyl Hydroxymethyl | Adeka Nol GE-RF | 14408-42-5 | Adeka Corporation |
| | Oleyl Oxazoline | | | |
| 95. | Pramoxine HCl | OriStar PMHCL | 637-58-1 | Orient Stars LLC |
| 96. | Allantoin Ascorbate | Allantoin Ascorbate | 57448-83-6 | ABI Chem |
| 97. | Stearamidopropyl | Mackalene ™ 326 | 55852-14-7 | Rhodia Inc. |
| | Morpholine Lactate | | | |
| 98. | Dioxolane | Elcotal DX | 646-06-0 | Lambiotte & CIE S.A. |
| 99. | Glycerol Formal | Glycerol Formal | 5464-28-8 | Sigma Aldrich (UK) |
| 100. | Stearamidopropyl | Mackine 321 | 55852-13-6 | Rhodia Inc. |
| | Morpholine | | | |
| 101. | 2,4,6-Tris[bis(methoxy- | Poly(melamine-co- | 68002-20-0 | Sigma-Aldrich (UK) |
| | methyl)amino]-1,3,5-triazine | formaldehyde) methylated | | |
| 102. | Poloxamine 1307 | Pluracare ® 1307 | 11111-34-5 | BASF |
| 103. | Nonoxynol-8 | Igepal ® CO-610 | 27177-05-5 | Rhodia Inc. |
| 104. | Nonoxynol-10 | Igepal ® CO-710 | 27177-08-8 | Rhodia Inc. |
| 105. | Octoxynol-10 | Nikkol OP-10 | 2315-66-4 | Nikko Chemicals Co., Ltd. |
| 106. | Nonoxynol-9 | Igepal ® CO-630 | 68987-90-6 | Rhodia Inc. |
| 107. | Nonoxynol-9 Iodine | Nonoxynol-9 iodine | 94349-40-3 | Angene Chemical |
| 108. | Octylphenoxy | Igepal ® CA-630 | 68987-90-6 | Rhodia Inc. |
| | poly(ethyleneoxy)ethanol, | | | |
| | branched | | | |
| 109. | Sodium Octoxynol-2 | Triton ™ X-200 | 55837-16-6 | The Dow Chemical Company |
| | Ethane Sulfonate | | | |
| 110. | Benzylhemiformal | Preventol D2 | 14548-60-8 | Lanxess Corporation |
| 111. | Nonoxynol-2 | Igepal ® CO-210 | 27176-93-8 | Rhodia Inc. |
| 112. | Octoxynol-3 | Igepal ® CA-420 | 2315-62-0 | The Dow Chemical Company |
| 113. | Nonoxynol-3 | Marlophen NP 3 | 27176-95-0 | Sasol Germany GmbH |
| 114. | Alkoxylated Alcohols | Alkasurf NP-4 | 7311-27-5 | Rhodia Inc. |
| 115. | Nonoxynol-3 | TriethyleneGlycol | 51437-95-7 | Santa Cruz Biotechnology |
| | | Mono(p-nonylphenyl) Ether | | |
| 116. | Nonoxynol-7 | Lowenol 2689 | 27177-03-3 | Jos. H. Lowenstein & Sons, Inc. |
| 117. | Nonoxynol-6 | Igepal ® CO-530 | 27177-01-1 | Rhodia Inc. |
| 118. | Nonoxynol-5 | Igepal ® CO-520 | 20636-48-0 | Rhodia Inc. |
| 119. | Nonoxynol-5 | Igepal ® CO-520 | 26264-02-8 | Rhodia Inc. |
| 120. | Nonoxynol-4 | Alkasurf NP-4 | 27176-97-2 | Rhodia Inc. |
| 121. | Polyglyceryl-10 Trioleate | Nikkol Decaglyn 3-OV | 102051-00-3 | Nikko Chemicals Co., Ltd. |
| 122. | Polyglyceryl-10 Dioleate | Nikkol Decaglyn 2-O | 33940-99-7 | Nikko Chemicals Co., Ltd. |
| 123. | Polyglyceryl-10 Tetraoleate | Caprol 10G40 | 34424-98-1 | Abitec Corporation |
| 124. | Polyglyceryl-10 Stearate | Nikkol Decaglyn 1-SV EX | 79777-30-3 | Nikko Chemicals Co., Ltd. |
| 125. | Polyglyceryl-10 Oleate | S-Face O-1001 P | 79665-93-3 | Sakamoto Yakuhin Kogyo Co., Ltd. |
| 126. | Polyglyceryl-10 Myristate | Nikkol Decaglyn 1-MV EV | 87390-32-7 | Nikko Chemicals Co., Ltd. |
| 127. | Dermofeel ® G 10 L | Dermofeel ® G 10 L | 34406-66-1 | Dr. Straetmans |
| 128. | Polyglyceryl-6 Laurate | NIKKOL Hexaglyn 1-L | 51033-38-6 | Chemical Navi |
| 129. | Polyglyceryl-6 Isostearate | S-Face IS-601 P | 126928-07-2 | Sakamoto Yakuhin Kogyo Co., Ltd. |
| 130. | Choleth-10 | Emalex CS-10 | 27321-96-6 | Nihon Emulsion Company, Ltd. |
| 131. | Steareth-10 Allyl Ether/ | Salcare ® SC80 | 109292-17-3 | BASF |
| | Acrylates Copolymer | | | |
| 132. | Polyvinyl Stearyl Ether | Giovarez ®1800 | 9003-96-7 | Phoenix Chemical, Inc. |
| 133. | Dicetyl Ether | Cosmacol Ether 16 | — | Sasol Germany GmbH |
| 134. | PPG-23-Steareth-34 | Unisafe 34S-23 | 9038-43-1 | Pola Chemical Industries, Inc. |
| 135. | Stearoxypropyl | Farmin DM E-80 | 17517-01-0 | Kao Corp. |
| | Dimethylamine | | | |
| 136. | Distearyl Ether | Cosmacol SE | 6297-03-6 | Sasol Germany GmbH |
| 137. | Polyquaternium-10 | AEC Polyquaternium-10 | 55353-19-0 | A & E Connock (Perfumery & |
| | | | | Cosmetics) Ltd. |
| 138. | Octyl ether | Dioctyl ether | 629-82-3 | Sigma Adlrich (UK) |
| 139. | Ethyl Ether | Diethyl Ether | 60-29-7 | EMD Chemicals |
| 140. | Methyl Hexyl Ether Ethers | methyl hexyl ether | 4747-07-3 | TCI AMERICA |
| 141. | Ceteth-12 | Emalex 112 | 94159-75-8 | Nihon Emulsion Company, Ltd. |
| 142. | Ceteth-10 or cetyl | Jeecol CA-10 | 14529-40-9 | Jeen International |
| | alcohol POE-10 | | | |
| 143. | Steareth-10 | Jeecol SA-10 | 13149-86-5 | Jeen International |
| 144. | Nonaethylene glycol | Nonaethylene glycol | 3055-99-0 | Sigma Aldrich (UK) |
| | monododecyl ether | monododecyl ether | | |
| 145. | Oleth-10 | Brij ® O10 | 71976-00-6 | Croda, Inc. |
| 146. | Oleth-10 | Brij ® O10 | 24871-34-9 | Croda, Inc. |
| 147. | PEG-12 | Carbowax ™ PEG 600 | 6790-09-6 | The Dow Chemical Company |
| 148. | PEG-9 | Sabopeg 400 | 3386-18-3 | Sabo s.p.a. |
| 149. | PEG-10 | DECAETHYLENE GLYCOL | 5579-66-8 | MolPort |
| 150. | PEG-6 | Carbowax ™ PEG 300 | 2615-15-8 | The Dow Chemical Company |
| 151. | Glycerol propoxylate | Glycerol propoxylate | 25791-96-2 | Sigma Aldrich (UK) |
| 152. | Glycerol ethoxylate | Glycerol ethoxylate | 31694-55-0 | Sigma Aldrich (UK) |
| 153. | Laureth-8 | AEC Laureth-8 | 3055-98-9 | A & E Connock (Perfumery & |
| | | | | Cosmetics) Ltd. |
| 154. | Oleth-8 | Emalex 508 | 27040-03-5 | Nihon Emulsion Company, Ltd. |
| 155. | Laureth-7 | Alfonic 1216CO-7 Ethoxylate | 3055-97-8 | Sasol North America, Inc. |
| 156. | Steareth-7 | Polyoxyethylene (7) stearyl | 66146-84-7 | Sigma Aldrich |
| | | ether | | |
| 157. | Deceth-6 | Alfonic 1012-6.0 Ethoxylate | 5168-89-8 | Sasol North America, Inc. |
| 158. | Steareth-6 | Emalex 606 | 2420-29-3 | Nihon Emulsion Company, Ltd. |
| 159. | Hexaethylene glycol | Hexaethylene glycol | 3055-96-7 | Sigma-Aldrich (UK) |
| | monododecyl ether | monododecyl ether | | |
| 160. | Hexaethylene glycol | Hexaethylene glycol | 5168-91-2 | Sigma-Aldrich (UK) |
| | monohexadecyl ether | monohexadecyl ether | | |
| 161. | Beheneth-5 | Nikkol BB-5 | 136207-49-3 | Nikko Chemicals Co., Ltd. |
| 162. | Myreth-5 | Isoxal 12 | 92669-01-7 | Vevy Europe SpA |
| 163. | Steareth-5 | Jeecol SA-5 | 71093-13-5 | Jeen International Corporation |
| 164. | Ceteth-5 | Emalex 105 | 4478-97-1 | Nihon Emulsion Company, Ltd. |
| 165. | Oleth-5 | Brij ® O5 | 5353-27-5 | Croda, Inc. |
| 166. | Laureth-5 | Safol ® 23E5 Ethoxylate | 3055-95-6 | Sasol North America, Inc. |
| 167. | Steareth-4 | Jeecol SA-4 | 59970-10-4 | Jeen International Corporation |
| 168. | Laureth-4 | Brij ® L4 | 5274-68-0 | Croda, Inc. |
| 169. | Myreth-4 | Homulgator 920 G | 39034-24-7 | Grau Aromatics GmbH & Company KG |
| 170. | Ceteth-4 | Procol CA-4 | 5274-63-5 | Protameen Chemicals |
| 171. | Oleth-4 | Chemal OA-4 | 5353-26-4 | Chemax, Inc. |
| 172. | Oleth-4 | Chemal OA-4 | 103622-85-1 | Chemax, Inc. |
| 173. | Polyimide-1 | Aquaflex ™ XL-30 | 497926-97-3 | Chemwill |
| 174. | Polymethoxy Bicyclic | Caswell No. 494CA | 56709-13-8 | Angene Chemical |
| | Oxazolidine | | | |
| 175. | Hydroxymethyl | Zoldine ™ ZT | 6542-37-6 | Angus Chemical Company |
| | Dioxoazabicyclooctane | | | |
| 176. | Dihydro-7a- | 5-Ethyl-1-aza- | 7747-35-5 | Sigma Aldrich (UK) |
| | ethyloxazolo[3,4-c]oxazole | 3,7-dioxabicyclo[3.3.0]octane | | |
| 177. | Dibenzylidene Sorbitol | Disorbene ® | 32647-67-9 | Roquette America, Inc. |
| 178. | Dimethyldibenzylidene | Millad ® 3988 | 135861-56-2 | Milliken Chemicals |
| | Sorbitol | | | |
| 179. | Laureth-2 | Alfonic 1216CO-2 Ethoxylate | 3055-93-4 | Sasol North America, Inc. |
| 180. | 2-(2-Butoxyethoxy)ethyl | Piperonyl Butoxide | 51-03-6 | Sigma-Aldrich (UK) |
| | (6-propylpiperonyl) ether | | | |
| 181. | Menthone Glycerin Acetal | Frescolat ® MGA | 63187-91-7 | Symrise |
| 182. | Propylene Glycol Caprylate | Mackaderm PGC | 68332-79-6 | Rhodia Inc. |
| 183. | Diethoxynonadiene | SBB016951 | 67674-36-6 | Ambinter |
| 184. | Menthoxypropanediol | Coolact ® 10 | 87061-04-9 | Takasago International Corporation |
| | Alcohols | | | |
| 185. | 2-Diphenylmethoxy- | Diphenhydramine HCl | 147-24-0 | Sigma-Aldrich (UK) |
| | N,N-dimethylethylamine | | | |
| | hydrochloride | | | |
| 186. | 3-((2-ethyl- | — | 70445-33-9 | — |
| | hexyl)oxy)propane-1,2-diol | | | |
| 187. | 3-((2-propyl- | — | — | — |
| | heptyl)oxy)propane-1,2-diol | | | |
| 188. | 1-amino-3-((2- | — | 99509-00-9 | — |
| | ethylhexyl)oxy)propan-2-ol | | | |
| 189. | 1-(1-Methyl-2- | Di(propylene glycol) | 29911-27-1 | Sigma Aldrich (UK) |
| | propoxyethoxy)-2-propanol | propyl ether | | |

According to some examples, the modulator(s) can be characterized as being “low odor”, “substantially non-odorous”, or non-odorous. In some examples, if the modulator is present at 1 wt % or less, no odor may be detected from the modulator.

In some examples, the modulator is biodegradable. This can make the composition to which it is included a “green” or environmentally friendly composition. Additionally, the stability of a composition that includes the substantially non-odorous modulator can be increased. The increase in stability can be shown by observing that the composition undergoes undesirable color formation (e.g., yellowing) at a slower rate.

The transparency component can help to make the transparent oil-in-water microemulsion optically clear or transparent and is present in a range of from about 5 wt % to about 20 wt % of the transparent oil-in-water microemulsion, about 10 wt % to about 15 wt %, less than, equal to, or greater than about 5 wt %, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or about 20 wt % of the transparent oil-in-water microemulsion. Examples of suitable transparency components can include diols. For example, certain diols can be vicinal diols, for example linear alkane vicinal diols, most preferably linear alkane vicinal diols having from 5 to 8 carbon atoms. For example the solvent may include 1,2-pentanediol; 1,2-hexanediol; 1,2-heptanediol; 1,2-octanediol; and mixtures thereof. Preferably, the solvent used in the present disclosure is 1,2-hexanediol. An example of a specific diol can include a 1,2-hexanediol.

Other ingredients that may optionally be present in the composition of the present disclosure may include for example antioxidants, chelating agents, UV filters, and preservatives. Additional ingredients such as thickening agents, cosmetic active ingredients, fragrance microcapsules, moisturizers, humectants, emollients, opacifiers, pearly gloss impacting substances, pigments, colorants, dyes and antifoams may also be optionally used in the composition of the present disclosure.

The optional additional ingredients are generally present in the composition of the present disclosure from about 0 wt % to about 5 wt %, preferably from about 0.5 wt % to about 3 wt %, and more preferably from about 0.1 wt % to about 1 wt %.

The transparent oil-in-water microemulsion can include and aqueous solvent. Specifically, the transparent oil-in-water microemulsion can include a water solvent that can range from about 10 wt % to about 95 wt % of the transparent oil-in-water microemulsion, about 40 wt % to about 60 wt %, less than, equal to, or greater than about 10 wt %, 15, 20, 25, 30, 35, 40, 45, 50, 55, 60, 65, 70, 75, 80, 85, 90, or about 95 wt % of the transparent oil-in-water microemulsion.

The transparent oil-in-water microemulsion according to the instant disclosure forms a microemulsion in which the transparent oil-in-water microemulsion is present in a series of small droplets surrounded by the surfactant component and bounded by water.

According to various examples, the composition can be a component of a kit. The kit can include a container. The transparent oil-in-water microemulsion can be disposed within the container.

### Examples

Various embodiments of the present disclosure can be better understood by reference to the following Examples which are offered by way of illustration. The present disclosure is not limited to the Examples given herein.

The following examples show various examples of transparent oil-in-water microemulsions that produced optically clear or transparent compositions.

A micro emulsion was created by mixing the components of Phase A and Phase B together at 800 RPM. Following mixing, additional component(s) were added until the solution was clear. Wt % values listed are of the total solution e.g., Phase A+Phase B+additional component(s).

TABLE 1

| | | | |
|---|---|---|---|
| INCI | Trade Name | Supplier | wt % |

Phase A

| | | | |
|---|---|---|---|
| Polyglyceryl-4 | Natragem S140 | Croda | 10-20 |
| Laurate/Sebacate (and) | | | |
| Polyglyceryl-6 | | | |
| Caprylate/Caprate | | | |
| (and) Aqua | | | |
| Propylene Glycol | Propylene Glycol | Spectrum | 3-8 |
| | | Chemical | |
| | | Mfg. Corp. | |
| Fragrance | | | 3-8 |
| Pentylene Glycol | Hydrolite 5 Green | Symrise | 3-8 |

Phase B

| | | | |
|---|---|---|---|
| Water | Water Deionized | n/a | 50-60 |
| GLDA | Dissolvine GL-47-S | Nouryon | 0.001-0.004 |

Additional components

| | | | |
|---|---|---|---|
| 1,2-Hexanediol | DL-1,2-Hexanediol | Acros | 8-12 |
| | | Organics | |
| Triethyl Citrate | Verstatil TBO | Evonik | 0.5-1.5 |
| (and) Caprylyl | | | |
| Glycol (and) | | | |
| Benzoic Acid | | | |

A micro emulsion was created by mixing the components of Phase A and Phase B together at 800 RPM. Following mixing, additional component(s) were added until the solution was clear. Wt % values listed are of the total solution e.g., Phase A+Phase B+additional component(s).

TABLE 2

| | | | |
|---|---|---|---|
| INCI | Trade Name | Supplier | wt % |

Phase A

| | | | |
|---|---|---|---|
| Polyglyceryl-4 | Natragem S140 | Croda | 15-25 |
| Laurate/Sebacate (and) | | | |
| Polyglyceryl-6 | | | |
| Caprylate/Caprate | | | |
| (and) Aqua | | | |
| Propylene Glycol | Propylene Glycol | Spectrum | 3-8 |
| | | Chemical | |
| | | Mfg. Corp. | |
| Fragrance | | | 8-12 |
| Pentylene Glycol | Hydrolite 5 Green | Symrise | 3-8 |

Phase B

| | | | |
|---|---|---|---|
| Water | Water Deionized | n/a | 40-50 |
| GLDA | Dissolvine GL-47-S | Nouryon | 0.001-0.004 |

Additional Component(s)

| | | | |
|---|---|---|---|
| 1,2-Hexanediol | DL-1,2-Hexanediol | Acros | 10-15 |
| | | Organics | |
| Triethyl Citrate | Verstatil TBO | Evonik | 0.5-1.5 |
| (and) Caprylyl | | | |
| Glycol (and) | | | |
| Benzoic Acid | | | |

A micro emulsion was created by mixing the components of Phase A and Phase B together at 800 RPM. Following mixing, additional component(s) were added until the solution was clear. Wt % values listed are of the total solution e.g., Phase A+Phase B+additional component(s).

TABLE 3

| | | | |
|---|---|---|---|
| INCI | Trade Name | Supplier | wt % |

Phase A

| | | | |
|---|---|---|---|
| Polyglyceryl- | Tegosoft | Evonik | 12-16 |
| 4 Caprate | PC 41 MB | | |
| Propylene | Propylene | Spectrum | 3-8 |
| Glycol | Glycol | Chemical Mfg. | |
| | | Corp. | |
| Fragrance | | | 3-8 |
| Pentylene | Hydrolite | Symrise | 3-8 |
| Glycol | 5 Green | | |

Phase B

| | | | |
|---|---|---|---|
| Water | Water | n/a | 60-70 |
| | Deionized | | |
| GLDA | Dissolvine | Nouryon | 0.001-0.004 |
| | GL-47-S | | |

Additional Component(s)

| | | | |
|---|---|---|---|
| 1,2- | DL-1,2- | Acros Organics | 5-10 |
| Hexanediol | Hexanediol | | |
| Triethyl | Verstatil | Evonik | 0.5-2 |
| Citrate (and) | TBO | | |
| Caprylyl | | | |
| Glycol (and) | | | |
| Benzoic Acid | | | |

A micro emulsion was created by mixing the components of Phase A and Phase B together at 800 RPM. Following mixing, additional component(s) were added until the solution was clear. Wt % values listed are of the total solution e.g., Phase A+Phase B+additional component(s).

TABLE 4

| | | | |
|---|---|---|---|
| INCI | Trade Name | Supplier | wt % |

Phase A

| | | | |
|---|---|---|---|
| Polyglyceryl-4 | Tegosoft PC | Evonik | 15-25 |
| Caprate | 41 MB | | |
| Propylene Glycol | Propylene | Spectrum | 3-8 |
| | Glycol | Chemical | |
| | | Mfg. Corp. | |
| Fragrance | | | 5-15 |
| Pentylene Glycol | Hydrolite | Symrise | 3-8 |
| | 5 Green | | |

Phase B

| | | | |
|---|---|---|---|
| Water | Water | n/a | 40-50 |
| | Deionized | | |
| GLDA | Dissolvine | Nouryon | 0.001-0.005 |
| | GL-47-S | | |

Additional Component(s)

| | | | |
|---|---|---|---|
| 1,2-Hexanediol | DL-1,2- | Acros | 10-15 |
| | Hexanediol | Organics | |
| Triethyl Citrate (and) | | Evonik | 0.5-1.5 |
| Caprylyl Glycol (and) | | | |
| Benzoic Acid | | | |

A micro emulsion was created by mixing the components of Phase A and Phase B together at 800 RPM. Following mixing, additional component(s) were added until the solution was clear. Wt % values listed are of the total solution e.g., Phase A+Phase B+additional component(s).

TABLE 5

| | | | |
|---|---|---|---|
| INCI | Trade Name | Supplier | wt % |

Phase A

| | | | |
|---|---|---|---|
| Polyglyceryl-4 | Natragem | Croda | 13-18 |
| Laurate/Sebacate (and) | S150 | | |
| Polyglyceryl-4 | | | |
| Caprylate/Caprate | | | |
| (and) Aqua | | | |
| Propylene Glycol | Propylene | Spectrum | 3-8 |
| | Glycol | Chemical | |
| | | Mfg. Corp. | |
| Fragrance | Summer | Firmenich | 3-7 |
| | Delight | | |
| Pentylene Glycol | Hydrolite 5 | Symrise | 3-8 |
| | Green | | |
| Phase B | | | |
| Water | Water | n/a | 40-60 |
| | Deionized | | |
| GLDA | Dissolvine | Nouryon | 0.001-0.004 |
| | GL-47-S | | |

Additional components

| | | | |
|---|---|---|---|
| 1,2-Hexanediol | DL-1,2- | Acros | 5-15 |
| | Hexanediol | Organics | |
| Triethyl Citrate (and) | Verstatil | Evonik | 0.5-1.5 |
| Caprylyl Glycol (and) | TBO | | |
| Benzoic Acid | | | |

A micro emulsion was created by mixing the components of Phase A and Phase B together at 800 RPM. Following mixing, additional component(s) were added until the solution was clear. Wt % values listed are of the total solution e.g., Phase A+Phase B+additional component(s).

TABLE 6

| | | | |
|---|---|---|---|
| INCI | Trade Name | Supplier | wt % |

Phase A

| | | | |
|---|---|---|---|
| Polyglyceryl-4 | Natragem | Croda | 15-25 |
| Laurate/Sebacate (and) | S150 | | |
| Polyglyceryl-4 | | | |
| Caprylate/Caprate (and) | | | |
| Aqua | | | |
| Propylene Glycol | Propylene | Spectrum | 3-8 |
| | Glycol | Chemical | |
| | | Mfg. Corp. | |
| Fragrance | | | 5-15 |
| Pentylene Glycol | Hydrolite 5 | Symrise | 3-9 |
| | Green | | |

Phase B

| | | | |
|---|---|---|---|
| Water | Water | n/a | 40-50 |
| | Deionized | | |
| GLDA | Dissolvine | Nouryon | 0.001-0.006 |
| | GL-47-S | | |

Additional Component(s)

| | | | |
|---|---|---|---|
| 1,2-Hexanediol | DL-1,2- | Acros | 10-15 |
| | Hexanediol | Organics | |
| Triethyl Citrate (and) | Verstatil TBO | Evonik | 0.5-2 |
| Caprylyl Glycol (and) | | | |
| Benzoic Acid | | | |

TABLE 7
A micro emulsion was created by mixing the components of
Phase A and Phase B together at 800 RPM. Following mixing,
additional component(s) were added until the solution
was clear. Wt % values listed are of the total solution
e.g., Phase A + Phase B + additional component(s).

| | | | |
|---|---|---|---|
| INCI | Tradename | Supplier | Wt % |

Phase A

| | | | |
|---|---|---|---|
| Polyglyceryl-6 Caprylate | Tegosolve | Evonik | 8-12 |
| Polyglyceryl-4 Caprate | 90 PC | | |
| Polyglyceryl-6 | Tegosolve | Evonik | 5-7 |
| Caprylate/Caprate | 61 | | |
| Polyglyceryl-3 Cocoate/ | | | |
| Polyglyceryl-4 Caprate/ | | | |
| Polyglyceryl-6 Ricinoleate | | | |
| (and) Aqua | | | |
| Propylene Glycol | Propylene | Spectrum | 3-8 |
| | Glycol | Chemical | |
| | | Mfg. Corp. | |
| Fragrance | | | 8-12 |
| Pentylene Glycol | Hydrolite | Symrise | 3-8 |
| | 5 Green | | |

Phase B

| | | | |
|---|---|---|---|
| Water | Water | n/a | 45-55 |
| | Deionized | | |
| GLDA | Dissolvine | Nouryon | 0.001-0.004 |
| | GL-47-S | | |

Additional Component(s)

| | | | |
|---|---|---|---|
| 1,2-Hexanediol | DL-1,2- | Acros | 8-12 |
| | Hexanediol | Organics | |
| | | check sum | 100.00 |

A micro emulsion was created by mixing the components of Phase A and Phase B together at 800 RPM. Following mixing, additional component(s) were added until the solution was clear. Wt % values listed are of the total solution e.g., Phase A+Phase B+additional component(s).

TABLE 8

| | | | |
|---|---|---|---|
| INCI | Supplier | Tradename | Wt % |

Phase A

| | | | |
|---|---|---|---|
| Polyglyceryl-6 Caprylate | Tegosolve | Evonik | 8-12 |
| Polyglyceryl-4 Caprate | 90 PC | | |
| Polyglyceryl-4 Caprylate | Tegosolve | Evonik | 5-7 |
| | PC 41 | | |
| Propylene Glycol | Propylene | Spectrum | 3-8 |
| | Glycol | Chemical | |
| | | Mfg. Corp. | |
| Fragrance | | | 8-12 |
| Pentylene Glycol | Hydrolite | Symrise | 3-8 |
| | 5 Green | | |

Phase B

| | | | |
|---|---|---|---|
| Water | Water | n/a | 45-55 |
| | Deionized | | |
| GLDA | Dissolvine | Nouryon | 0.001-0.004 |
| | GL-47-S | | |

Additional Component(s)

| | | | |
|---|---|---|---|
| 1,2-Hexanediol | DL-1,2- | Acros Organics | 8-12 |
| | Hexanediol | | |
| | | check sum | 100.00 |

A micro emulsion was created by mixing the components of Phase A and Phase B together at 800 RPM. Following mixing, additional component(s) were added until the solution was clear. Wt % values listed are of the total solution e.g., Phase A+Phase B+additional component(s).

TABLE 9

| | | | |
|---|---|---|---|
| INCI | Trade Name | Supplier | wt % |

Phase A

| | | | |
|---|---|---|---|
| Polyglyceryl-6 Caprylate | Tegosolve | Croda | 10-20 |
| Polyglyceryl-4 Caprate | 90 | | |
| Hydrogenated Lecithin (and) | Biophilic | Lucas Meyer | 0.1-1 |
| C12-16 Alcohols (and) | H MB | Cosmetics | |
| Palmitic Acid | | | |
| Propylene Glycol | Propylene | Spectrum | 3-8 |
| | Glycol | Chemical | |
| | | Mfg. Corp. | |
| Fragrance | | | 3-8 |
| Pentylene Glycol | Hydrolite | Symrise | 3-8 |
| | 5 Green | | |

Phase B

| | | | |
|---|---|---|---|
| Water | Water | n/a | 50-60 |
| | Deionized | | |
| GLDA | Dissolvine | Nouryon | 0.001-0.004 |
| | GL-47-S | | |

Additional components

| | | | |
|---|---|---|---|
| 1,2-Hexanediol | DL-1,2- | Acros | 8-12 |
| | Hexanediol | Organics | |
| | | Check sum | 100.00 |

The fragrance compositions represented in Tables 1-9 are expected to perform better than a comparative (e.g., control) fragrance composition that includes ethanol as a solvent. In a comparative fragrance composition, the ethanol can range from about 60 wt % to about 70 wt % of the composition. The comparative fragrance compositions will not include water.

The superior, and unexpected, performance of the fragrance compositions in Tables 1-9 is that they are expected to be free of a harsh perception by a panel of experts or professional evaluators or individual experts or professional evaluators. The panel of experts or professional evaluators or individual experts or professional evaluators will evaluate according to the following protocol. At the testing facility, 50 μL samples of the compositions and the controls are applied to glass slides and placed on a hot plate at 32° C. to represent skin temperature for varying durations. It is important that glass slides of samples that are to be later compared are prepared at the same time. The panelists are asked to evaluate the perceived fragrance profile (intensity and/or character) of each glass slide sample at a given time point. Slides are presented coded so that their identity is not known by the panelists. Within a given time point panelists evaluate the slides in a random order and are able to revisit their assessment as they work through the slides at that time point. Their assessments are recorded. In the subsequent analysis, the data for strength and character comparisons are drawn from the independent assessments carried out at a given time point. Only when using the character difference scale below are any 2 products physically directly compared to each other. Panelists are selected from individuals who are either trained to evaluate fragrances according to the scales below or who have experience of fragrance evaluation in the industry. Typically, around 4-6 panelists are used to evaluate a given product and its control.

### (a) Fragrance Intensity:

The panelists will be asked to give a score on a scale of 0 to 5 for perceived fragrance intensity according to the odor intensity scale set out in Table 10 herein below.

TABLE 10
Odor Intensity Scale

| Score | Fragrance Intensity |
|---|---|
| 0 | None |
| 1 | Very Weak |
| 2 | Weak |
| 3 | Moderate |
| 4 | Strong |
| 5 | Very Strong |

### (b) Fragrance Character:

The panelists will be asked to assess the fragrance character in one of 2 ways:
i) a score on a scale of 0 to 3 for the dominance of particular characters that are relevant to that particular fragrance, e.g.: fresh, harsh, green, watery, floral, rose, muguet, fruity, apple, berry, citrus, creamy, woody, balsamic, amber, musk just to name a few, according to the odour grading scale set out in Table 6(i) herein below;
ii) a score on a scale of 1 to 5 for changes in the perceived fragrance profile change for the test compositions versus the controls according to the odour grading scale set out in Table 6(ii) herein below.

TABLE 11
Character Dominance Odor Grading Scale

| Score | Fragrance Character Dominance |
|---|---|
| 0 | Not noticeable |
| 1 | Slight presence of the character |
| 2 | Moderate presence of the character |
| 3 | Dominance of the character |

TABLE 12
Character Difference Odor Grading Scale

| Score | Fragrance Profile Change |
|---|---|
| 1 | Fragrance profile is unchanged, i.e., no difference |
| | between the sample vs. the control |
| 2 | Slight fragrance profile change when compared directly |
| | with the control |
| 3 | Moderate fragrance profile but similar character to the |
| | control, |
| 4 | Large difference in fragrance profile from the control. |
| 5 | Total difference in the fragrance profile from the control. |

The results of the panelists are averaged and if sufficient panelists are available, typically around 10, then the data can be analyzed using Analysis of Variance methods. The model treats the subject as a random effect and looks at the impact of product, time and the interaction between product and time. From the analysis the least square means for the product and time interaction are obtained. These means (as well as their confidence intervals) are then plotted to enable comparisons between products at each time point. It should be noted that the confidence levels plotted are intended as a guide, and not as a statistical comparison, as they do not take into account that multiple testing has been performed. As well as a graphical assessment, statistical comparisons between the two products at each of the time points are performed with a Sidak correction for multiple comparisons. The p-values for the product differences are obtained, with p-values <0.05 indicating a statistical difference between the two products at 5% significance (or 95% confidence). Typically for assessments by expert panelists of evaluators and perfumers there are 4-6 participants. In these cases a full statistical analysis is not possible and typically we observe that an average difference of 0.75 on the scales used considered to be meaningful, i.e.: 3 out of 4 experts gave the products a grade with a difference of 1.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present disclosure. Thus, it should be understood that although the present disclosure has been specifically disclosed by specific embodiments and optional features, modification and variation of the concepts herein disclosed may be resorted to by those of ordinary skill in the art, and that such modifications and variations are considered to be within the scope of embodiments of the present disclosure.

### Exemplary Embodiments

The following exemplary embodiments are provided, the numbering of which is not to be construed as designating levels of importance:

Aspect 1 provides a transparent oil-in-water microemulsion composition comprising:
a fragrance component;
a surfactant;
an optional modulator, comprising a glycol or polycitronellol;
water; and
a transparency component, wherein
the transparent oil-in-water microemulsion comprises less than 3 wt % ethanol.

Aspect 2 provides the transparent oil-in-water microemulsion of Aspect 1, wherein the fragrance component is in a range of from about 0.5 wt % to about 15 wt % of the transparent oil-in-water microemulsion.

Aspect 3 provides the transparent oil-in-water microemulsion of any one of Aspects 1 or 2, wherein the fragrance component is in a range of from about 5 wt % to about 12 wt % of the transparent oil-in-water microemulsion.

Aspect 4 provides the transparent oil-in-water microemulsion of any one of Aspects 1-3, wherein the transparent oil-in-water microemulsion comprises musk oil, civet, castoreum, ambergris, plant fragrances such as nutmeg extract, cardomon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomile oil, clove oil, sage oil, neroli oil, labdanum oil, eucalyptus oil, verbena oil, mimosa extract, narcissus extract, carrot seed extract, jasmine extract, olibanum extract, rose extract, acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, benzyl acetate, benzyl alcohol and ester derivatives, benzyl propionate, benzyl salicylate, borneol, butyl acetate, camphor, carbitol, cinnamaldehyde, cinnamyl acetate, cinnamyl alcohol, cis-3-hexanol and ester derivatives, cis-3-hexenyl methyl carbonate, citral, citronellol and ester derivatives, cumin aldehyde, cyclamen aldehyde, cyclo galbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltolide, fenchone, fruity esters such as ethyl 2-methyl butyrate, galaxolide, geraniol and ester derivatives, helional, 2-heptonone, hexenol, α-hexylcinnamic aldehyde, hydroxycitronellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dihydrojasmonate, methyl eugenol, methyl ionone, methyl-β-naphthyl ketone, methylphenylcarbinyl acetate, mugetanol, γ-nonalactone, octanal, phenyl ethyl acetate, phenyl-acetaldehyde dimethyl acetate, phenoxyethyl isobutyrate, phenyl ethyl alcohol, pinenes, sandalore, santalol, stemone, thymol, terpenes, triplal, triethyl citrate, 3,3,5-trimethylcyclohexanol, γ-undecalactone, undecenal, vanillin, veloutone, verdox, or mixtures thereof.

Aspect 5 provides the transparent oil-in-water microemulsion of any one of Aspects 1-4, wherein the surfactant is in a range of from about 10 wt % to about 25 wt % of the transparent oil-in-water microemulsion.

Aspect 6 provides the transparent oil-in-water microemulsion of any one of Aspects 1-5, wherein the surfactant is in a range of from about 14 wt % to about 20 wt % of the transparent oil-in-water microemulsion.

Aspect 7 provides the transparent oil-in-water microemulsion of any one of Aspects 1-6, wherein the surfactant comprises a polyethylene glycol-free surfactant.

Aspect 8 provides the transparent oil-in-water microemulsion of Aspect 7, wherein the polyethylene glycol-free surfactant comprises a polyglyceryl fatty acid ester, a sugar based surfactant, or a mixture thereof

Aspect 9 provides the transparent oil-in-water microemulsion of Aspect 8, wherein the polyethylene glycol-free surfactant comprises polyglyceryl fatty acid ester.

Aspect 10 provides the transparent oil-in-water microemulsion of Aspect 9, wherein the polyglyceryl fatty acid ester comprises:
a mixture of:
   polyglyceryl-4 laurate/sebacate; polyglyceryl-6 caprylate/caprate; and water,
   polyglyceryl-4 laurate/sebacate, polyglyceryl-4 caprylate/caprate; or
   polyglyceryl-4 caprate.
   Polyglyceryl-6 Caprylate, Polyglyceryl-4 Caprate
   Polyglyceryl-3 Cocoate, Polyglyceryl-6 Ricinoleate

Aspect 11 provides the transparent oil-in-water microemulsion of any one of Aspects 1-10, wherein the transparent oil-in-water microemulsion is a microemulsion and the microemulsion is formed by the surfactant encasing the fragrance component.

Aspect 12 provides the transparent oil-in-water microemulsion of any one of Aspects 1-11, wherein the modulator is present in a range of from about 1 wt % to about 15 wt % of the transparent oil-in-water microemulsion.

Aspect 13 provides the transparent oil-in-water microemulsion of any one of Aspects 1-12, wherein the modulator is present in a range of from about 3 wt % to about 10 wt % of the transparent oil-in-water microemulsion.

Aspect 14 provides the transparent oil-in-water microemulsion of any one of Aspects 1-13, wherein the modulator comprising glycol, comprises pentylene glycol, glucam, or a mixture thereof.

Aspect 15 provides the transparent oil-in-water microemulsion of any one of Aspects 1-14, wherein the modulator comprising glycol comprises pentylene glycol.

Aspect 16 provides the transparent oil-in-water microemulsion of any one of Aspects 1-15, wherein the transparency component is present in a range of from about 5 wt % to about 20 wt % of the transparent oil-in-water microemulsion.

Aspect 17 provides the transparent oil-in-water microemulsion of any one of Aspects 1-16, wherein the transparency component is present in a range of from about 8 wt % to about 15 wt % of the transparent oil-in-water microemulsion.

Aspect 18 provides the transparent oil-in-water microemulsion of any one of Aspects 1-17, wherein the transparency component comprises a diol.

Aspect 19 provides the transparent oil-in-water microemulsion of any one of Aspects 1-17, wherein the transparency component comprises a 1,2-hexanediol.

Aspect 20 provides the transparent oil-in-water microemulsion of any one of Aspects 1-19, wherein the transparent oil-in-water microemulsion is a perfume, an Eaux de Toilette, an aqueous perfume, a body spray, or a body deodorant.

Aspect 21 provides a kit comprising:
a container; and
the transparent oil-in-water microemulsion of any one of Aspects 1-20, disposed within the container.
   Aspect 22 provides a transparent oil-in-water microemulsion comprising:
a fragrance component in a range of from about 0.5 wt % to about 15 wt % of the transparent oil-in-water microemulsion;
a surfactant in a range of from about 10 wt % to about 20 wt % of the transparent oil-in-water microemulsion, the surfactant comprising a mixture of:
   polyglyceryl-4 laurate/sebacate; polyglyceryl-6 caprylate/caprate; and water,
   polyglyceryl-4 laurate/sebacate, polyglyceryl-4 caprylate/caprate; and water,
or
   polyglyceryl-4 caprate;
   Polyglyceryl-6 Caprylate, Polyglyceryl-4 Caprate
   Polyglyceryl-3 Cocoate, Polyglyceryl-6 Ricinoleate
a modulator present in a range of from about 1 wt % to about 15 wt % of the transparent oil-in-water microemulsion, the modulator comprising pentylene glycol, glucam, or a mixture thereof;
water; and
a transparency component present in a range of from about 5 wt % to about 20 wt % of the transparent oil-in-water microemulsion the transparency component comprising a 1,2-hexanediol, wherein
the transparent oil-in-water microemulsion comprises less than 1 wt % ethanol.

1. A transparent oil-in-water microemulsion composition comprising:
a fragrance component;
a surfactant;
an modulator, comprising a pentylene glycol polycitronellol, a mixture thereof;
water; and
a transparency component, wherein
the transparent oil-in-water microemulsion comprises less than 3 wt % ethanol.

2. The transparent oil-in-water microemulsion of claim 1, wherein the fragrance component is in a range of from about 0.5 wt % to about 15 wt % of the transparent oil-in-water microemulsion.

3. The transparent oil-in-water microemulsion of claim 1, wherein the fragrance component is in a range of from about 5 wt % to about 12 wt % of the transparent oil-in-water microemulsion.

4. The transparent oil-in-water microemulsion of claim 1, wherein the transparent oil-in-water microemulsion comprises musk oil, civet, castoreum, ambergris, plant fragrances such as nutmeg extract, cardomon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomile oil, clove oil, sage oil, neroli oil, labdanum oil, eucalyptus oil, verbena oil, mimosa extract, narcissus extract, carrot seed extract, jasmine extract, olibanum extract, rose extract, acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, benzyl acetate, benzyl alcohol and ester derivatives, benzyl propionate, benzyl salicylate, borneol, butyl acetate, camphor, carbitol, cinnamaldehyde, cinnamyl acetate, cinnamyl alcohol, cis-3-hexanol and ester derivatives, cis-3-hexenyl methyl carbonate, citral, citronellol and ester derivatives, cumin aldehyde, cyclamen aldehyde, cyclo galbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltolide, fenchone, fruity esters such as ethyl 2-methyl butyrate, galaxolide, geraniol and ester derivatives, helional, 2-heptonone, hexenol, α-hexylcinnamic aldehyde, hydroxycitronellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dihydrojasmonate, methyl eugenol, methyl ionone, methyl-β-naphthyl ketone, methylphenylcarbinyl acetate, mugetanol, γ-nonalactone, octanal, phenyl ethyl acetate, phenyl-acetaldehyde dimethyl acetate, phenoxyethyl isobutyrate, phenyl ethyl alcohol, pinenes, sandalore, santalol, stemone, thymol, terpenes, triplal, triethyl citrate, 3,3,5-trimethylcyclohexanol, γ-undecalactone, undecenal, vanillin, veloutone, verdox, or mixtures thereof.

5. The transparent oil-in-water microemulsion of claim 1, wherein the surfactant is in a range of from about 10 wt % to about 25 wt % of the transparent oil-in-water microemulsion.

6. The transparent oil-in-water microemulsion of any one of claim 1, wherein the surfactant is in a range of from about 14 wt % to about 20 wt % of the transparent oil-in-water microemulsion.

7. The transparent oil-in-water microemulsion of claim 1, wherein the surfactant comprises a polyethylene glycol-free surfactant.

8. The transparent oil-in-water microemulsion of claim 7, wherein the polyethylene glycol-free surfactant comprises a polyglyceryl fatty acid ester, a sugar based surfactant, or a mixture thereof

9. The transparent oil-in-water microemulsion of claim 8, wherein the polyethylene glycol-free surfactant comprises polyglyceryl fatty acid ester.

10. The transparent oil-in-water microemulsion of claim 9, wherein the polyglyceryl fatty acid ester comprises:
a mixture of:
polyglyceryl-4 laurate/sebacate; polyglyceryl-6 caprylate/caprate; and water,
polyglyceryl-4 laurate/sebacate, polyglyceryl-4 caprylate/caprate; and water, or
polyglyceryl-4 caprate.
Polyglyceryl-6 Caprylate, Polyglyceryl-4 Caprate
Polyglyceryl-3 Cocoate, Polyglyceryl-6 Ricinoleate

11. The transparent oil-in-water microemulsion of claim 1, wherein the transparent oil-in-water microemulsion is a microemulsion and the microemulsion is formed by the surfactant encasing the fragrance component.

12. The transparent oil-in-water microemulsion of claim 1, wherein the modulator is present in a range of from about 1 wt % to about 15 wt % of the transparent oil-in-water microemulsion.

13. The transparent oil-in-water microemulsion of claim 1, wherein the modulator is present in a range of from about 3 wt % to about 10 wt % of the transparent oil-in-water microemulsion.

14. (canceled)

15. (canceled)

16. The transparent oil-in-water microemulsion of claim 1, wherein the transparency component is present in a range of from about 5 wt % to about 20 wt % of the transparent oil-in-water microemulsion.

17. The transparent oil-in-water microemulsion of claim 1, wherein the transparency component is present in a range of from about 8 wt % to about 15 wt % of the transparent oil-in-water microemulsion.

18. The transparent oil-in-water microemulsion of claim 1, wherein the transparency component comprises a diol.

19. The transparent oil-in-water microemulsion of claim 1, wherein the transparency component comprises a 1,2-hexanediol.

20. The transparent oil-in-water microemulsion of claim 1, wherein the transparent oil-in-water microemulsion is a perfume, an Eaux de Toilette, an aqueous perfume, a body spray, or a body deodorant.

21. A kit comprising:
a container; and
the transparent oil-in-water microemulsion of claim 1, disposed within the container.

22. A transparent oil-in-water microemulsion comprising:
a fragrance component in a range of from about 0.5 wt % to about 15 wt % of the transparent oil-in-water microemulsion;
a surfactant in a range of from about 10 wt % to about 20 wt % of the transparent oil-in-water microemulsion, the surfactant comprising a mixture of:
polyglyceryl-4 laurate/sebacate; polyglyceryl-6 caprylate/caprate; and water,
polyglyceryl-4 laurate/sebacate, polyglyceryl-4 caprylate/caprate; and water, or
polyglyceryl-4 caprate;
Polyglyceryl-6 Caprylate, Polyglyceryl-4 Caprate
Polyglyceryl-3 Cocoate, Polyglyceryl-6 Ricinoleate
a modulator present in a range of from about 1 wt % to about 15 wt % of the transparent oil-in-water microemulsion, the modulator comprising pentylene glycol, polycitronellol, glucam, or a mixture thereof;
water; and
a transparency component present in a range of from about 5 wt % to about 20 wt % of the transparent oil-in-water microemulsion the transparency component comprising a 1,2-hexanediol, wherein
the transparent oil-in-water microemulsion comprises less than 1 wt % ethanol.
